(19) United States
(12) Patent Application Publication
(10) Pub. No.: US 2025/0040807 A1
(43) Pub. Date: Feb. 6, 2025

(54) SYSTEM AND METHOD FOR ALIGNMENT OF A HANDHELD DEVICE WITH RESPECT TO AN EYE IMPLANT

(71) Applicant: Twenty Twenty Therapeutics LLC, South San Francisco, CA (US)

(72) Inventors: Supriyo Sinha, Menlo Park, CA (US); Oleg Rumyantsev, St. Johns, FL (US); Dimitri Azar, Chicago, IL (US)

(21) Appl. No.: 18/362,458

(22) Filed: Jul. 31, 2023

(51) Int. Cl. A61B 3/16; G06T 7/00; G06T 7/60; G06T 7/70

(52) U.S. Cl. CPC A61B 3/16; G06T 7/0012; G06T 7/60; G06T 7/70; G06T 2207/30041

(57) ABSTRACT

A system for spatially aligning an external measuring device and an intraocular implant, the system comprising: an intraocular implant configured to be implanted into an eye of a user to monitor or measure intraocular parameters; and an external measuring device configured to receive and measure the intraocular parameters when aligned with the intraocular implant, the external measuring device comprising one or more visual targets and one or more mirrors operable to align a line of sight of the eye so that the intraocular implant is aligned with a main optical path of the external measuring device for measuring.

### FIELD

The subject matter of this disclosure relates to techniques for aligning a measuring device with an intraocular implant in an eye to measure intraocular parameters. Such techniques are useful for treating and/or monitoring progression of eye diseases including glaucoma, but are not limited to use with the treatment of eye disease.

### BACKGROUND

Typically, intraocular parameters within an eye, for example intraocular pressure (IOP), are measured using a tonometer. A tonometer is a device that is outside the eye and thus does not require a sensor within the eye. Contact tonometry is performed in a clinical setting, and the procedure requires numbing of the patient's eye, resulting in both inconvenience and discomfort. Noncontact tonometry involves directing a puff or jet of air towards the patient's eye and measuring the resulting deflection dynamics of the cornea. However, this requires a bulky and power hungry pump arrangement that may not be practical for home use, and is not as accurate as contact tonometry. In addition, in some instances, it is desirable to be able to measure such parameters frequently and from the convenience of ones home. For example, in the case of a condition such as glaucoma, which causes chronic heightened IOP, treatment mainly involves periodically administering pharmaceutical agents to the eye to decrease IOP. These drugs can be delivered, for example, by injection or eye drops. However, effective treatment of glaucoma requires adherence to dosage schedules and a knowledge of the patient's IOP. The IOP for a given patient can vary significantly based on time of day, exercise, how recently a medication was taken, and other factors. Typically, IOP measurements are performed in a doctor's office and often no more than once or twice per year. These infrequent measurements are less able to account for variation in the patient's IOP, and may become stale due to the length of time between them. This means that any given measurement is subject to uncertainty, so it may take several IOP measurements over time to have confidence in the health of the patient's eye.

A wireless, implantable, continuous IOP monitoring system has been suggested that has a commercial pressure sensing element with digital readout, and a microelectronic chip that supports wireless power/data telemetry and a wired serial communication interface with the pressure sensing element. An on-chip integrated RF coil receives power from near-field RF coupling at 915 MHz, and transmits pressure measurement bits via RF-backscattering to an external reader. To ensure accurate measurements by the external reader, however, it is critical that the external reader be precisely aligned with the pressure sensing element.

### SUMMARY

An aspect of the disclosure is a system and process for aligning a measuring device with an intraocular implant in an eye to measure intraocular parameters. The general principle is the following. The user holds a handheld device and moves it towards their eye while trying to look at visual targets associated with, or integral to, the handheld device. Alignment can be passive (i.e., no feedback generated by the handheld device) or active. In the latter case, the user may receive visual/audio or haptic feedback based on optimal/suboptimal alignment. This, in turn, is determined by the strength of the handheld device-implant coupling or by computer vision system (e.g., a camera mounted on a handheld device tracking the angle and distance to the eye). In one aspect, in order to achieve proper angular alignment, the user needs to overlap two targets by looking at and/or through them.

Similar to a sight device, this process allows angular alignment of the eye (and therefore an implant) with a handheld device. In order to also provide proper distance, the visual targets (e.g., objects/apertures/pinholes) may have different sizes, so that at proper distance from the eye, their angular size is the same. Another distance positioning or determining technique may be implemented using a computer vision algorithm. For example, a camera mounted on a handheld device may track the pupil size and/or orientation which, in turn, can be converted into distance to the eye. At a correct or desired predetermined distance, a visual/audio cue informs the user of correct alignment and optical measurement may then begin. Positioning of targets, apertures, pinholes, guiding light sources, etc., can coincide with the main optical path of the measuring device, in which case the path used for alignment may be combined with the main optical path using a dichroic mirror. This also requires spectral separation of light used for optical measurement and visual cues used for alignment. Once proper separation between the pupil and the device is achieved and direction of sight corresponds with the main optical axis, the user may saccade to a specified target (e.g., a lighting light emitting diode (LED)) or order. This, in turn, puts the implant on the optical axis of the measuring device. In some aspects, for compactness, the optical path used for alignment may be folded by use of mirrors. Due to variability of implant position and angle, specific positions of elements aiding the alignment (e.g., position of LED to saccade to) may be adjusted for individual users. Elements which may be used as visual targets for alignment may include, but are not limited to, pinholes, apertures, targets on semi-transparent film/glass, illuminated features or patterns, light sources (e.g., LEDs), miniature reflecting or dichroic mirrors, or a larger mirror combined with an aperture, in which case reflection of the user's pupil becomes a target itself. The main advantage of the systems and processes disclosed herein is that they allow performing non-contact optical readout of a signal from an implant in an efficient and easy (from the user's perspective) way.

Representatively, in one aspect, a system for spatially aligning an external measuring device and an intraocular implant includes an intraocular implant configured to be implanted into an eye of a user to monitor or measure intraocular parameters; and an external measuring device configured to receive and measure the intraocular parameters when aligned with the intraocular implant, the external measuring device comprising one or more visual targets and one or more mirrors operable to align a line of sight of the eye so that the intraocular implant is aligned with a main optical path of the external measuring device for measuring. In one aspect, the one or more visual targets includes a first target and a second target that are offset from the main optical path and operable to be co-aligned with the line of sight, and the one or more mirrors comprises a first mirror operable to fold the line of sight and a second mirror operable to combine the line of sight with the main optical path. In another aspect, the one or more mirrors includes a dichroic mirror aligned with the main optical path and the one or more visual targets comprises an aperture aligned with the main optical path and a reflection of a pupil of the user's eye through the aperture by the dichroic mirror. In still further aspects, the one or more visual targets include an object and an aperture vertically offset from the main optical path, and aligning the line of sight with the aperture and the object aligns the intraocular implant with the main optical path. In some aspects, aligning the line of sight includes a first alignment in which the line of sight is aligned with the main optical path, and a second alignment in which the line of sight is aligned with a second target offset from the main optical path so that the intraocular implant is aligned with the main optical path. In some aspects, the external measuring device further includes a distance determining mechanism for detecting an optimal distance between the eye and the external measuring device for measuring. In some aspects, the distance determining mechanism includes a camera that tracks a size or orientation of a pupil of the eye to determine the distance. In some aspects, the distance determining mechanism includes a computerized mechanism that implements a computer vision algorithm to determine the distance.

In other aspects, a system for spatially aligning an external measuring device and an intraocular implant includes an intraocular implant configured to be implanted into an eye of a user to monitor intraocular parameters; an external measuring device configured to receive and measure the intraocular parameters when aligned with the intraocular implant; and one or more processors communicatively coupled to the external measuring device and configured to: receive alignment data indicating an alignment of the eye relative to a main optical path of the external measuring device; receive distance data indicating a distance of the external measuring device to the eye; and determine, based on the alignment data and the distance data, whether the external measuring device is spatially aligned with the intraocular implant such that receiving and measuring of the intraocular parameters may proceed. In some aspects, the external measuring device further includes one or more visual targets and one or more mirrors operable to align a line of sight of the eye such that the intraocular implant is aligned with the main optical path. In some aspects, the one or more visual targets include a first target and a second target that are offset from the main optical path and operable to be co-aligned with the line of sight, and the one or more mirrors comprises a first mirror operable to fold the line of sight and a second mirror operable to combine the line of sight with the main optical path. In still further aspects, the one or more mirrors include a dichroic mirror aligned with the main optical path and the one or more visual targets comprises an aperture aligned with the main optical path and a pupil of the user's eye reflected by a dichroic mirror through the aperture. In some aspects, the alignment is a first alignment indicating the line of sight is aligned with the main optical path, and the alignment data further comprises a second alignment indicating the line of sight is aligned with a second target offset from the main optical path, and wherein aligning the line of sight with the second target aligns the intraocular implant with the main optical path. In still further aspects, the distance data indicates a distance between a pupil of the eye and the external measuring device, and the processor determines whether the distance is optimal for measuring by the external measuring device. In some aspects, the external measuring device includes one or more visual targets having different sizes, and the visual targets appear to have a same size when the distance between the pupil and the external measuring device is optimal for measuring. In still further aspects, the external measuring device includes an optical instrument for tracking a size or orientation of the pupil of the eye of the user to determine the distance.

In still further aspects, a method for spatially aligning an external measuring device with an implant in an eye of a user includes detecting an alignment of a line of sight of the eye of the user relative to a main optical path of an external measuring device positioned near the eye of the user for measuring of an intraocular parameter obtained by the implant; tracking, using an optical instrument, a distance of the external measuring device, to the eye of a user; and determining, by one or more processors communicatively coupled to the external measuring device, based on the aligning and the tracking, whether the implant is aligned with the main optical path and the distance is optimal for measuring by the external measuring device. In some aspects, detecting alignment includes detecting a first alignment of the line of sight of the eye with a first target, a second target, and the main optical path using a dichroic mirror; and detecting a second alignment of the line of sight in which the line of sight is adjusted to align with a third target offset from the main optical path to spatially align the implant with the main optical path. In some aspects, alignment includes detecting an alignment of the line of sight of the eye with one or more visual targets offset from the main optical path, and wherein when the line of sight is aligned with the one or more visual targets, the implant is aligned with the main optical path. In some aspects, the optical instrument is a camera, and tracking comprises tracking a pupil size and orientation by the camera to determine the distance.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings, in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1A shows a schematic illustration of an example system for measuring an intraocular parameter of an eye.

FIG. 1B shows a magnified view of an implant for monitoring the intraocular parameter implanted into a sclera of the eye of FIG. 1A.

FIG. 2 shows a schematic view of a user holding a handheld device close to their eye for measuring an intraocular parameter of the eye of FIG. 1A.

FIGS. 3A-3B show block diagrams illustrating an example system for aligning a handheld device with an implant for measuring an intraocular parameter.

FIGS. 4A-4B show block diagrams illustrating an example system for aligning a handheld device with an implant for measuring an intraocular parameter.

FIG. 5 is a block diagram illustrating an example system for aligning a handheld device with an implant for measuring an intraocular parameter.

FIG. 6 is a block diagram illustrating an example system for measuring an intraocular parameter.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

The terminology used herein is for the purpose of describing particular aspects only and is not intended to be limiting of the disclosure. Spatially relative terms, such as “beneath”, “below”, “lower”, “above”, “upper”, and the like may be used herein for ease of description to describe one element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (e.g., rotated 90 degrees or at other orientations) and the spatially relative descriptors used herein interpreted accordingly.

As used herein, the singular forms “a”, “an”, and “the” are intended to include the plural forms as well, unless the context indicates otherwise. It will be further understood that the terms “comprises” and/or “comprising” specify the presence of stated features, steps, operations, elements, and/or components, but do not preclude the presence or addition of one or more other features, steps, operations, elements, components, and/or groups thereof.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

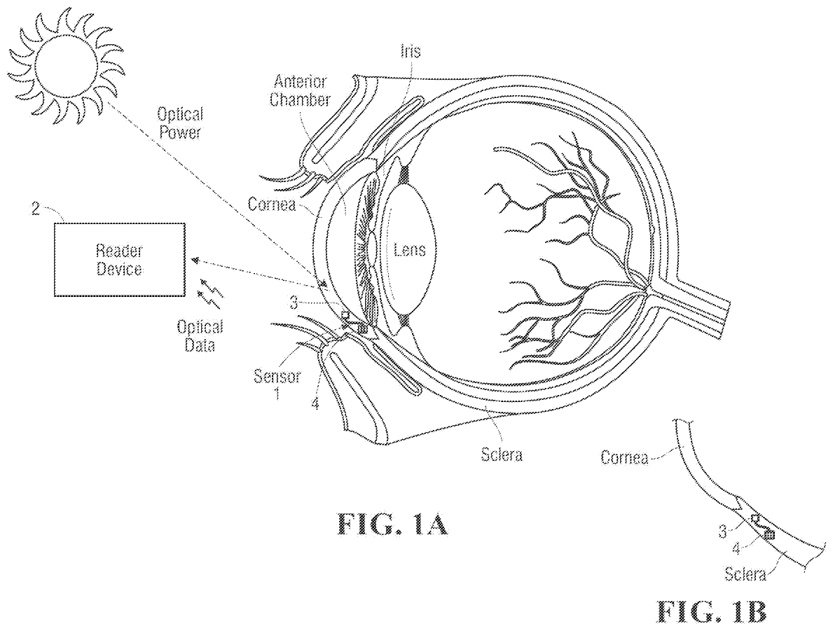

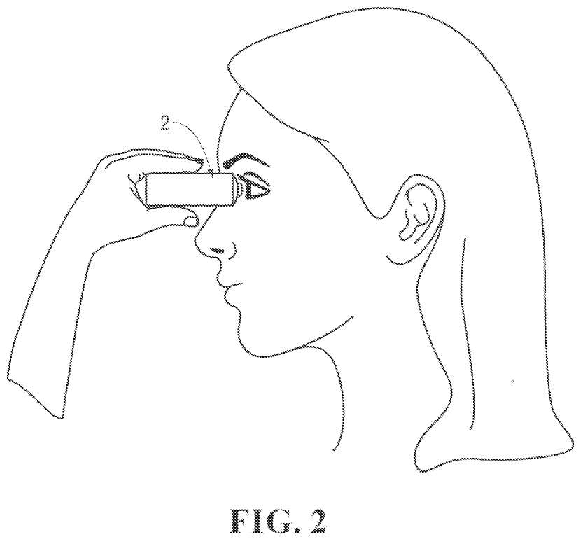

FIG. 1A and FIG. 1B show an example system for measuring an intraocular parameter, using a handheld device 2 (e.g., a reader device) and an implant 1 (e.g., IOP sensor). The implant 1 may be composed of several parts or components which cooperate to enable the implant 1 to have an all optical interface, e.g., an optical power interface and an optical data interface. Note that some of these parts are depicted in a block diagram of the system shown in FIG. 6. In FIG. 1A, the implant is implanted in its entirety within the cornea, while in FIG. 1B the implanted in its entirety within the sclera.

Implant 1 may generally include the following components. An intraocular sensing element 3 is to be implanted into an eye of a user, in the cornea (e.g., as shown in FIG. 1A) or in the sclera (e.g., as shown in FIG. 1B.) The intraocular pressure sensing element 3 may be, for example, a capacitive or piezoelectric pressure sensing element. The sensing element may be conductively coupled to implant microelectronic circuitry 4 which is also implanted into the eye, in the cornea as shown in the example of FIG. 1A or in the sclera as shown in the example of FIG. 1B. This conductive coupling may be through a connection between a housing of the intraocular sensing element 3 and a housing of the implant microelectronic circuitry 4. Alternatively, the connection may be a flexible one like a tether which allows the two components (the intraocular sensing element 3 and the housing that contains the implant microelectronic circuitry 4 and perhaps other components described below) to be positioned at separately optimal locations in the eye.

Implant microelectronic circuitry 4 uses a signal (in the conductive coupling) from the intraocular sensing element 3 to produce measured data. The signal may be an analog signal (e.g., that is input to a sensing amplifier which is part of the implant microelectronic circuitry 4), or it may be a digital signal (e.g., the sensing amplifier and a digitizer are part of the intraocular pressure sensing element 3.) The implant microelectronic circuitry 4 drives a microscopic light emitting diode, micro LED 6 (see FIG. 6), the latter also being implanted in the eye of the user for example within the same housing or package as the implanted microelectronic circuitry 4. The micro LED 6 is thus optically transmitting the measured intraocular parameter data, for communication with outside of the eye (e.g., to the handheld device 2). For example, the micro LED 6 may transmit light at a wavelength that is in the visible region or in the near infrared region. The micro LED 6 may be no more than 0.2 millimeters squared in area. In some aspects, the implant 1 may also include a photovoltaic element 7 and a battery 8 as shown in FIG. 6. The photovoltaic element 7 may be used to convert incident ambient light, e.g., solar, into energy that it supplies to operate the implant and the battery 8 may be charged and recharged by the photovoltaic element 7. In addition, in some aspects, the implant microelectronic circuitry 4 may include an application specific integrated circuit, ASIC, that has a watchdog timer that triggers the taking of measurements of the implant (or producing measured pressure data) at intervals programmed into the watchdog timer. In one aspect, this allows the implant 1 to take measurements at night time or when the user is asleep.

FIG. 2 depicts a user holding the handheld device 2 in their hand, and who has brought the handheld device close to their eye. In this aspect, the handheld device 2 is a portable or handheld device in which outside-the-eye microelectronic circuitry has been integrated. The outside-the-eye microelectronic circuitry is configured to receive the transmitted, measured intraocular data once the handheld device 2 is close to the eye and aligned with implant 1, for example using or more LEDs or other suitable photodetector arrangement. For example, the handheld device 2 may be deemed aligned with implant 1 when device 2 determines that certain alignment parameters are met and/or alignment operations are performed (e.g., visual targets are aligned), and close to the eye when it can receive the optically transmitted data (e.g., no more than three inches away from the eye). Once device 2 determines that alignment and distance parameters are met, device 2 can then receive and process the measured pressure data from implant 1 and inform the user about the measured intraocular parameter.

For example, in one aspect, the handheld device outside-the-eye microelectronic circuitry transmits an optical interrogation signal that is detected by the implant microelectronic circuitry using the microscopic LED (operating in reverse bias), the photovoltaic element, or a separate photodetector element. In response, the implant microelectronic circuitry becomes aware that the handheld device 2 is aligned and within range to receive data from implant 1, and therefore drives the micro LED with the measured data thereby optically transmitting the measured data to the outside-of the-eye microelectronic circuitry of device 2. The outside-of the-eye microelectronic circuitry may also include or be associated with one or more processors or processing components that, based on alignment and distance data, can also determine device 2 is spacially aligned and within range of implant 1 to receive and process the measured parameters. In some aspects, a memory within implant 1 stores or logs all intraocular parameters that are monitored and/or measured to be read by the handheld device 2.

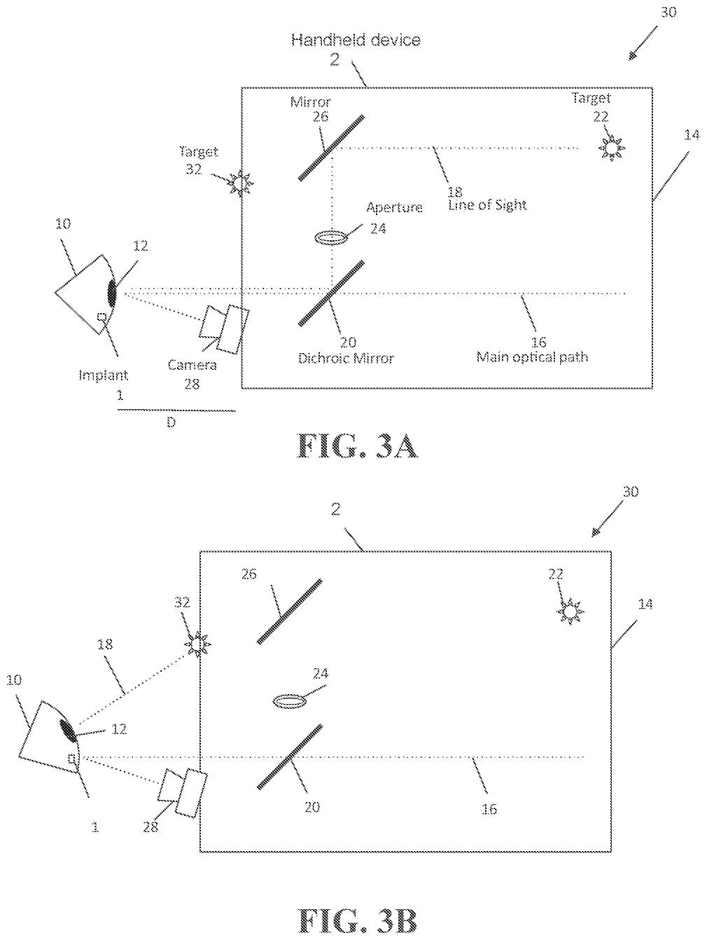

FIGS. 3A-3B illustrate schematic diagrams of one representative system for aligning a handheld device with an implant for measuring an intraocular parameter. In one aspect, system 30 includes handheld device 2 for reading or measuring the intraocular parameters monitored by implant 1. Representatively, implant 1 may be an implant that is implanted within eye 10 of the user (e.g., a cornea or sclera of the eye of the user) as previously discussed in reference to FIG. 1A or FIG. 1B. In order for handheld device 2 to perform optical measurement of parameters measured by implant 1 and/or perform optical read/write of data/instructions, precise spatial alignment and distance between handheld device 2 and implant 1 is necessary. Therefore, in some aspects, handheld device 2 may include a housing 14 having a number of components integrated therein for achieving the necessary special alignment and distance between handheld device 2 and implant 1. Once a proper alignment is achieved, handheld device 2 may read and/or measure the intraocular parameters monitored/measured by implant 1.

Representatively, a number of alignment and/or distance detecting components may be integrated into handheld device 2 for facilitating proper alignment and positioning of handheld device 2 relative to the eye 10 and/or implant 1 needed for measuring. In some aspects, the alignment components may include one or more mirrors 20, 26 and visual targets 22, 24 for aligning a line of sight 18 so that the implant 1 is aligned with a main optical path 16 of handheld device 1 for measuring.

Representatively, in some aspects, handheld device 2 may include a main optical path 16 which must be aligned with implant 1 for the intraocular parameters monitored and/or measured by implant 1 to be read by handheld device 2. For example, optical path 16 may be, for example, a laser beam or other type of optical path suitable for aligning implant 1 with handheld device 2. A dichroic mirror 20 may further be aligned with optical path 16 at an angle as shown in FIGS. 3A-3B. The dichroic mirror 20 may allow the main optical path 16 to pass through, while reflecting visible light, as shown. In this aspect, the user's line of sight 18 may be aligned with visual targets 22, 24, and then combined with main optical path 16 using the dichroic mirror 20. For example, the visual targets 22, 24 may include an aperture 24 and an object 22 that are offset from the main optical path 16. The user's line of sight 18 is aligned with object 22 by looking at and aligning the aperture 24 with object 22. Mirror 26 may further be positioned within handheld device 2 as shown to fold line of sight 18 for compactness. For example, mirror 26 may be angled and vertically aligned with dichroic mirror 20 as shown so that even though the aperture 24 and object 20 are vertically offset from the main optical path 16, they can be co-aligned by the eye 10, and the line of sight 18 can be aligned (or combined) with the main optical path 16. Alignment of line of sight 18 with the main optical path 16, aligns pupil 12 of eye 10 relative to handheld device 2. In other words, pupil 12 is aligned with main optical path 16. Since the location of implant 1 relative to pupil 12 is known, knowing the position of pupil 12 relative to main optical path 16 allows the system to further determine a position of implant 1 relative to handheld device 2. For example, it can be seen from FIG. 3A that when pupil 12 is aligned with main optical path 12, implant 1 is slightly below main optical path 12. In addition, it should be understood that due to variability of implant position and angle, specific positions of elements aiding the alignment (e.g. position of targets) may be adjusted for individual users.

Once a proper angular alignment of eye 10 (and therefore implant 1) relative to device 2 is determined, an optimal distance (D) between the pupil 12 and device 2 is determined. Representatively, to ensure accurate measurement, the eye 10 (and therefore implant 1) should be at a distance (D) from device 2 that is optimal for the implant microelectronic circuitry to communicate the measured or monitored data optically, e.g.,

via a microscopic light emitting diode (micro LED) that transmits the data, out of the eye. For example, one representative optimal distance (D) may be three inches or less. System 30 may therefore also include a distance determining mechanism for monitoring or determining whether eye 10 is a desired distance from device 2. Representatively, in one aspect, device 2 may have an optical instrument 28 for tracking a pupil size and/or orientation. This, in turn, can be converted into distance to the eye. For example, optical instrument 28 may be a camera 28 that tracks the pupil size and/or orientation. This information may, in turn, be processed by the device to determine whether eye 10 is a proper distance from device 2. In other aspects, visual targets (e.g., visual targets 22, 24) may be used to determine a proper distance. For example, the visual targets may have different sizes. Moving eye 10 closer or farther away from device 2 may cause the sizes of the visual targets to appear the same. When the sizes of the visual targets appear the same, eye 10 is determined to be at the proper distance from device 2. In still further aspects, the distance determining mechanism may implement a computer vision algorithm which uses obtained visual data to determine a proper distance. The alignment and distance information or data obtained or determined by device 2 may be used (e.g., processed) to determine whether the handheld device-implant alignment and/or distance is suitable to proceed with measuring. In some aspects, the user may receive visual, audio or haptic feedback from device 2 indicating whether the alignment and/or distance are optimal or suboptimal for measuring, and/or whether measuring may proceed. The

In some aspects, once an initial alignment and distance between eye 10 and device 2 are confirmed, the user saccades eye 10 to another target 32 (or order) to ultimately align implant 1 with main optical path 16 for measuring. Representatively, as illustrated by FIG. 3B, eye 10 saccades upward toward a second target 32 (e.g., a lighting LED) so that line of sight 18 is now aligned with target 32. This, in turn, moves implant 1 upward so that it is now aligned with main optical path 16 for measuring. Implant 1 is now at a proper angular orientation and distance relative to handheld device 2 and measuring can now occur. It should be understood that the term “saccade” refers to a rapid movement of the eye between two or more points. Any type of movement, or shift, from the first target 22 to the second target 32, however, is contemplated.

System 30 may further include a processor or processing component (see e.g., processor 9 of FIG. 6) which is configured to detect and/or otherwise receive and process the alignment and/or distance data. For example, the alignment and/or distance data may be detected, determined, measured or otherwise obtained by a sensor (e.g., a motion sensor using IR technology), tracking or other component of device 2 suitable for obtaining the alignment and/or distance data disclosed herein. Based on this data, the processor may then determine whether device 2 is spatially aligned with implant 1 such that a measuring operation may proceed.

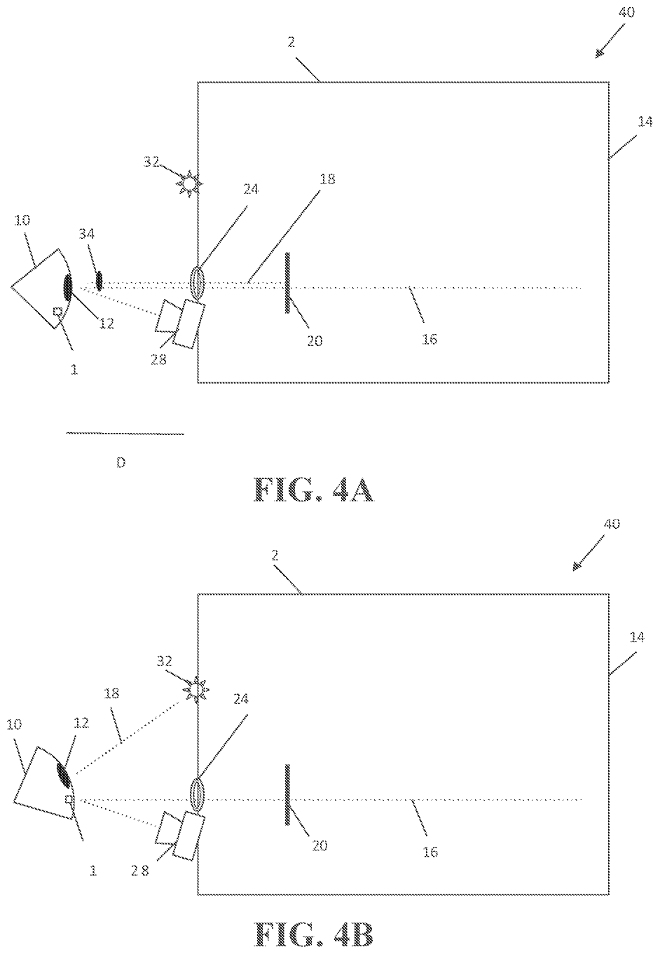

FIGS. 4A-4B illustrate schematic diagrams of one representative system for aligning a handheld device with an implant for measuring an intraocular parameter. System 40 is substantially similar to the system described in reference to FIGS. 3A-3B and includes handheld device 2 for reading and/or measuring the intraocular parameters monitored and/or measured by implant 1. Representatively, handheld device 2 may include a housing 14 having a number of components integrated therein for achieving the necessary special alignment and distance between handheld device 2 and implant 1. Once a proper alignment and distance is achieved, handheld device 2 may read and/or measure the intraocular parameters monitored/measured by implant 1.

Representatively, a number of alignment and/or distance detecting components may be integrated into handheld device 2 for facilitating proper alignment and positioning of handheld device 2 relative to the eye 10 and/or implant 1. For example, the alignment components may include a mirror 20 and visual target 24 for aligning a line of sight 18 relative to a main optical path 16 of handheld device 1, as previously discussed in reference to FIGS. 3A-3B. In this configuration, however, the user's pupil 12 is used as a target therefore an additional target and mirror may be omitted. Representatively, dichroic mirror 20 and target 24 may be aligned with main optical path 16 so that its reflective surface is perpendicular to path 16. Target 24 may be an aperture or ring that is aligned at its center with path 16. In this aspect, when the user's pupil 12 is properly aligned with main optical path 16 in front of mirror 20, the user sees a reflection of their pupil 12 through target 24. The reflection of pupil 12 through aperture 24 therefore serves as the target that is used to align the user's line of sight 18 with main optical path 16. As previously discussed, since the location of implant 1 relative to pupil 12 is known, knowing the position of pupil 12 relative to main optical path 16 allows the system to further determine a position of implant 1 relative to handheld device 2. For example, it can be seen from FIG. 4A that when pupil 12 is aligned with main optical path 12, implant 1 is slightly below main optical path 12.

Once a proper angular alignment of the eye 10 (and therefore implant 1) relative to device 2 is determined, a proper distance between the pupil 12 and device 2 is determined. System 40 may therefore also include components such as those previously discussed for positioning eye 10 a desired distance from device 2. Representatively, in one aspect, to ensure a proper distance, visual targets (e.g., objects, apertures, pinholes, etc.) positioned along the line of sight 18 may have different sizes. Moving the eye 10 closer or farther away from device 2 may cause the sizes of the visual targets to appear the same. When the sizes of the visual targets appear the same, the eye 10 is at the proper distance from device 2. Another distance positioning technique may implement a computer vision algorithm used to confirm a proper distance. Still further, in some aspects, device 2 may have an optical instrument 28 for tracking a pupil size and/or orientation. This, in turn, can be converted into distance to the eye. For example, optical instrument 28 may be a camera 28 that tracks the pupil size and/or orientation. This information may, in turn, be processed by the device to determine whether eye 10 is a proper distance from device 2.

In some aspects, once an initial alignment and distance between eye 10 and device 2 are confirmed, the user saccades eye 10 to target 32 (or order) to ultimately align implant 1 with main optical path 16 for measuring. Representatively, as illustrated by FIG. 4B, eye 10 saccades upward toward target 32 (e.g., a lighting LED) so that line of sight 18 is now aligned with target 32. This, in turn, moves implant 1 upward so that it is now aligned with main optical path 16 for measuring. Implant 1 is now at a proper angular orientation and distance relative to handheld device 2 and measuring can now occur.

System 40 may further include a processor or processing component (see e.g., processor 9 of FIG. 6) which is configured to detect and/or otherwise receive and process the alignment and/or distance data. For example, the alignment and/or distance data may be communicated as an alignment or distance optical readout signal corresponding to the parameters measured and/or monitored by the implant 1. Based on this data, the processor may then determine whether device 2 is properly aligned with implant 1 such that a measuring operation may proceed.

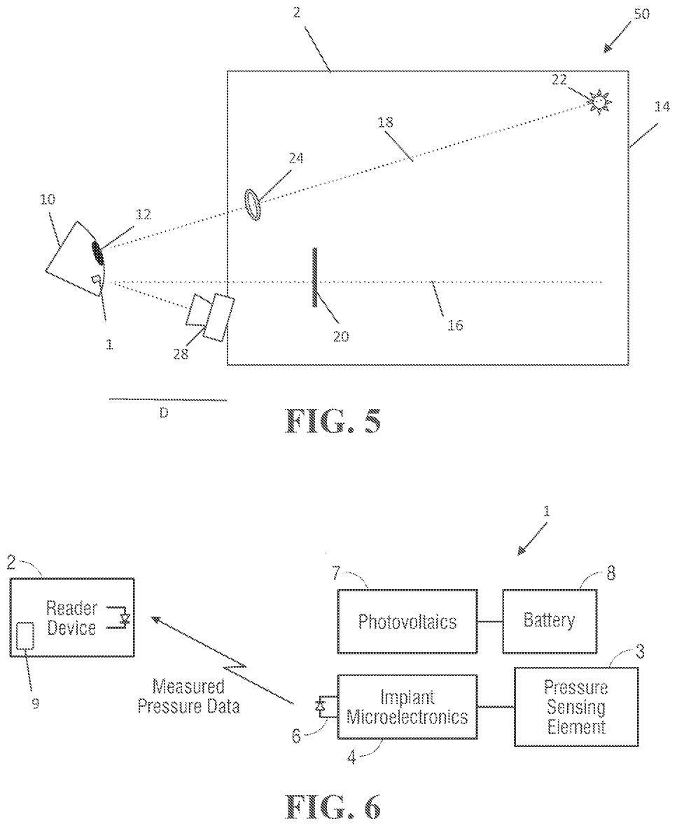

FIG. 5 illustrates a schematic diagram of one representative system for aligning a handheld device with an implant for measuring an intraocular parameter. System 50 is substantially similar to the system described in reference to FIGS. 3A-3B and includes handheld device 2 for reading and/or measuring the intraocular parameters monitored and/or measured by implant 1. Representatively, handheld device 2 may include a housing 14 having a number of components integrated therein for achieving the necessary special alignment and distance between handheld device 2 and implant 1. Once a proper alignment and distance is achieved, handheld device 2 may read and/or measure the intraocular parameters monitored/measured by implant 1.

Representatively, a number of alignment and/or distance detecting components may be integrated into handheld device 2 for facilitating proper alignment and positioning of handheld device 2 relative to the eye 10 and/or implant 1. For example, the alignment components may include a mirror 20 and visual targets 22, 24 for aligning a line of sight 18 relative to a main optical path 16 of handheld device 1, as previously discussed in reference to FIGS. 3A-3B. In this configuration, however, the line of sight 18 is initially aligned with targets 22, 24 in such a way that it aligns implant 1 with main optical path 16, without an additional mirror and/or saccading the eye to another target. Representatively, dichroic mirror 20 may be aligned with main optical path 16 so that its reflective surface is perpendicular to path 16. The target 22 is offset and above main optical path 16 at a particular location so that when the user co-aligns targets 22, 24 with eye 10, implant 1 is aligned with main optical path 16 as shown in FIG. 5. As previously discussed, since the location of implant 1 relative to pupil 12 is known. Accordingly, target 22 can be positioned at a particular location such that when line of sight 18 from pupil 12 is aligned with targets 22, 24, implant 1 is aligned with main optical path 12.

Once a proper angular alignment of the eye 10 (and therefore implant 1) relative to device 2 is determined, a proper distance between the pupil 12 and device 2 is determined. System 50 may therefore also include components such as those previously discussed for positioning eye 10 a desired distance from device 2. Representatively, in one aspect, to ensure a proper distance, visual targets 22, 24 (e.g., objects, apertures, pinholes, etc.) positioned along the line of sight 18 may have different sizes. Moving the eye 10 closer or farther away from device 2 may cause the sizes of the visual targets to appear the same. When the sizes of the visual targets appear the same, the eye 10 is at the proper distance from device 2. Alternatively, the user may move handheld device 2 relative to eye 10 until the size of the targets 22, 24 are the same. Another distance positioning technique may implement a computer vision algorithm used to confirm a proper distance. Still further, in some aspects, device 2 may have an optical instrument 28 for tracking a pupil size and/or orientation. This, in turn, can be converted into distance to the eye. For example, optical instrument 28 may be a camera 28 that tracks the pupil size and/or orientation. This information may, in turn, be processed by the device to determine whether eye 10 is a proper distance from device 2.

System 50 may further include a processor or processing component (see e.g., processor 9 of FIG. 6) which is configured to detect and/or otherwise receive and process the alignment and/or distance data. For example, the alignment and/or distance data may be communicated as an alignment or distance optical readout signal corresponding to the parameters measured and/or monitored by the implant 1. Based on this data, the processor may then determine whether device 2 is properly aligned with implant 1 such that a measuring operation may proceed.

While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. For example, it should be understood that while targets such as objects and apertures are described and shown in the drawings, any suitable type of target is contemplated. Representatively, other elements which may be used as targets, may include, but are not limited to, pinholes, apertures, targets on semi-transparent film/glass, illuminated features or patterns, light sources (e.g., LEDs), miniature reflecting or dichroic mirrors, or a large mirror combined with an aperture, in which case reflection of the user's pupil becomes the target itself. The description is thus to be regarded as illustrative instead of limiting.

What is claimed is:

1. A system for spatially aligning an external measuring device and an intraocular implant, the system comprising:
an intraocular implant configured to be implanted into an eye of a user to monitor or measure intraocular parameters; and
an external measuring device configured to receive and measure the intraocular parameters when aligned with the intraocular implant, the external measuring device comprising one or more visual targets and one or more mirrors operable to align a line of sight of the eye so that the intraocular implant is aligned with a main optical path of the external measuring device for measuring.

2. The system of claim 1 wherein the one or more visual targets comprises a first target and a second target that are offset from the main optical path and operable to be co-aligned with the line of sight, and the one or more mirrors comprises a first mirror operable to fold the line of sight and a second mirror operable to combine the line of sight with the main optical path.

3. The system of claim 1 wherein the one or more mirrors comprises a dichroic mirror aligned with the main optical path and the one or more visual targets comprises an aperture aligned with the main optical path and a reflection of a pupil of the user's eye through the aperture by the dichroic mirror.

4. The system of claim 1 wherein the one or more visual targets comprises an object and an aperture vertically offset from the main optical path, and aligning the line of sight with the aperture and the object aligns the intraocular implant with the main optical path.

5. The system of claim 1 wherein aligning the line of sight comprises a first alignment in which the line of sight is aligned with the main optical path, and a second alignment in which the line of sight is aligned with a second target offset from the main optical path so that the intraocular implant is aligned with the main optical path.

6. The system of claim 1 wherein the external measuring device further comprises a distance determining mechanism for detecting an optimal distance between the eye and the external measuring device for measuring.

7. The system of claim 6 wherein the distance determining mechanism comprises a camera that tracks a size or orientation of a pupil of the eye to determine the distance.

8. The system of claim 6 wherein the distance determining mechanism comprises a computerized mechanism that implements a computer vision algorithm to determine the distance.

9. A system for spatially aligning an external measuring device and an intraocular implant, the system comprising:
an intraocular implant configured to be implanted into an eye of a user to monitor intraocular parameters;
an external measuring device configured to receive and measure the intraocular parameters when aligned with the intraocular implant; and
one or more processors communicatively coupled to the external measuring device and configured to:
receive alignment data indicating an alignment of the eye relative to a main optical path of the external measuring device;
receive distance data indicating a distance of the external measuring device to the eye; and
determine, based on the alignment data and the distance data, whether the external measuring device is spatially aligned with the intraocular implant such that receiving and measuring of the intraocular parameters may proceed.

10. The system of claim 9 wherein the external measuring device further comprises one or more visual targets and one or more mirrors operable to align a line of sight of the eye such that the intraocular implant is aligned with the main optical path.

11. The system of claim 10 wherein the one or more visual targets comprises a first target and a second target that are offset from the main optical path and operable to be co-aligned with the line of sight, and the one or more mirrors comprises a first mirror operable to fold the line of sight and a second mirror operable to combine the line of sight with the main optical path.

12. The system of claim 10 wherein the one or more mirrors comprises a dichroic mirror aligned with the main optical path and the one or more visual targets comprises an aperture aligned with the main optical path and a pupil of the user's eye reflected by a dichroic mirror through the aperture.

13. The system of claim 9 wherein the alignment is a first alignment indicating the line of sight is aligned with the main optical path, and the alignment data further comprises a second alignment indicating the line of sight is aligned with a second target offset from the main optical path, and wherein aligning the line of sight with the second target aligns the intraocular implant with the main optical path.

14. The system of claim 9 wherein the distance data indicates a distance between a pupil of the eye and the external measuring device, and the processor determines whether the distance is optimal for measuring by the external measuring device.

15. The system of claim 14 wherein the external measuring device comprises one or more visual targets having different sizes, and the visual targets appear to have a same size when the distance between the pupil and the external measuring device is optimal for measuring.

16. The system of claim 14 wherein the external measuring device comprises an optical instrument for tracking a size or orientation of the pupil of the eye of the user to determine the distance.

17. A method for spatially aligning an external measuring device with an implant in an eye of a user comprising:
detecting an alignment of a line of sight of the eye of the user relative to a main optical path of an external measuring device positioned near the eye of the user for measuring of an intraocular parameter obtained by the implant;
tracking, using an optical instrument, a distance of the external measuring device, to the eye of a user; and
determining, by one or more processors communicatively coupled to the external measuring device, based on the aligning and the tracking, whether the implant is aligned with the main optical path and the distance is optimal for measuring by the external measuring device.

18. The method of claim 17 wherein detecting alignment comprises:
detecting a first alignment of the line of sight of the eye with a first target, a second target, and the main optical path using a dichroic mirror; and
detecting a second alignment of the line of sight in which the line of sight is adjusted to align with a third target offset from the main optical path to spatially align the implant with the main optical path.

19. The method of claim 17 wherein alignment comprises:
detecting an alignment of the line of sight of the eye with one or more visual targets offset from the main optical path, and wherein when the line of sight is aligned with the one or more visual targets, the implant is aligned with the main optical path.

20. The method of claim 17 wherein the optical instrument is a camera, and tracking comprises tracking a pupil size and orientation by the camera to determine the distance.
